(19) United States
(12) Patent Application Publication
(10) Pub. No.: US 2006/0293613 A1
(43) Pub. Date: Dec. 28, 2006

(54) Method and Apparatus for Automated Monitoring and Tracking of the Trajectory of Patients' Center of Gravity Movements

(75) Inventors: Mohammad Taghi Fatehi, Irvine, CA (US); Leslie Alan Baxter, Little Silver, NJ (US)

Correspondence Address: CONCEPT DEVELOPMENT GROUP, 7 ALMOND TREE LANE, IRVINE, CA, 92612, US

(73) Assignee: CONCEPT DEVELOPMENT GROUP, Irvine, CA (US)

(21) Appl. No.: 11/160,513

(22) Filed: Jun. 27, 2005

(51) Int. Cl. A61B 5/103; A61B 5/117

(52) U.S. Cl. 600/587; 600/595

(57) ABSTRACT

This application describes a revolutionary device to help prevent the development of pressure ulcers. The device works by using a small number of sensors in the wheelchair or bed to determine and track the projection of the patient's center of gravity (COP) onto the plane of the chair or bed. The sensor data are processed by various algorithms which determine when the danger of pressure ulcer development arises and alerts an attendant to reposition the patient. The repositioning is likewise monitored, and if it is inadequate, a higher-level alarm is issued. This device also maintains a history of patient position and movement, which is potentially useful for diagnostic purposes, such as tracking the degree of improvement resulting from a course of treatment or therapy. The archival record can also provide verification of proper care in the event of lawsuits or insurance claims. Operation of the device is non-intrusive and does not disturb either the patient or the caregiver unless repositioning of the patient is needed. The invention is based on simple, fundamental principles of physics and engineering. It is implemented with a few force sensors, microprocessors, and wireless networking technology—all of which are readily available. The unique attributes of this apparatus are: Real time performance coupled with maintenance of an archival history. Inexpensive hardware platform. Fully self-contained apparatus. Flexible algorithms for processing the sensor data and tracking motion trajectories in a quantitative manner. The hardware portion of the apparatus may adapted to be placed in the wheelchair or bed under the normal cushions, for mobility-impair patients, or it may be used as a force platform, much like a bathroom scale, for standing stability monitoring or gait training. It transmits the instantaneous COP position information over a wired or wireless network to a processing and monitoring station. It does not employ any external components such as video cameras, local GPS transmitters, etc. Aside from a tool for prevention of pressure in mobility-impaired patients, various embodiments of this system has applications in physical and occupational therapy, gait training, mental training (biofeedback), sports and athletic training, (e.g., golf swing training, baseball batting practice), gymnastics, yoga, dance/ballet, sports paraphernalia design, and a host of other areas, etc.

This application is a non-provisional conversion of the provisional patent application previously filed with USPTO: Application No. 60/583,544, USPTO Filing Date Jun. 29, 2004, Confirmation No. 4399, under the same title.

### TECHNICAL FIELD OF THE INVENTION

This invention relates to the field of physical medicine, rehabilitation and biomedical engineering describing the method and apparatus for automated monitoring and tracking of the trajectory of body movements in mobility-impaired patients, tracking the stability of mentally-unstable or intoxicated individuals, tracking the course of therapy, and as an aid for physical and mental training; and more specifically for prevention of pressure ulcers in mobility-impaired patients, and quantitative analysis of the degree of stability in able-bodied individuals.

The invention also has numerous other applications in non-medical fields such as sobriety testing, sports training, etc.

### BACKGROUND OF THE INVENTION

Around ten thousand cases are added each year to an estimated quarter to a half million individuals living with Spinal Cord Injury (SCI) or Spinal Dysfunction in the US, according to the National Spinal Cord Injury Association Resource Center^{[1,2]}. The major causes of these are motor vehicle accidents, acts of violence, falls, and sports injuries. Damage caused by acute SCI—namely, contusion (bruising) or transection (tearing) of the spinal cord—can result in decreased (or complete disappearance of) movement, sensation, and body organ function below the level of the injury.

While SCI is a common cause of permanent disability, other causes include sudden afflictions such as stroke^{[3,4]} (relatively common), as well as debilitating progressive diseases such as multiple sclerosis^{[5]}. Regrettably (and recently high-profile), “man-made” causes also exist, including combat wounds suffered by military personnel^{[6]}.

Physically disabled wheelchair-bound individuals (such as paraplegic and quadriplegic people) as well as bed-bound patients such as stroke patients are prone to developing pressure-induced ulcers commonly known as decubitus ulcers—or in common parlance, bedsores^{[7,8,9]}. Since stricken individuals in the population of interest lack the required sensory system in addition to motor abilities, they must rely on trained health care professionals or others to be moved and repositioned from time to time (every one to three hours) in order to prevent the occurrence of bedsores.

It is to be emphasized that bedsores are not to be dismissed trivially. They are akin to burns, and if not properly attended to, can progress to the point of causing severe infection and become life-threatening. If unchecked, the tissue destruction can lead to the necessity of amputation in advanced stages. Clearly, bedsores are a serious matter.

As bedsores are as old as medical care itself, naturally a variety of palliative and ameliorative treatments have been developed and are available on the market, in both conventional and alternative arenas^{[10,11]}. However, nowhere has the homily “prevention is better than cure” been more true than in the case of bedsores^{[12]}. Perhaps the most compelling argument for prevention is the fact that the occurrence of bedsores is viewed (with just cause) as being caused by negligence of the care provider, and prone to trigger hue and cry, and in extreme cases, lawsuits.

Examination of prior art reveals while there is no dearth of proposed means to prevent the occurrence of bedsores^{[13-21]}, not all have been implemented as commercially available products due to practical or cost considerations. Special mattresses and wheelchair cushions are on the market^{[22-24]}, and while these claim to providing relief; none of these is an effective substitute for a ministering attendant, and/or are very expensive. Pressure mapping systems that utilize multitude of pressure/force sensors pads are commercially available^{[25]}, but this technology is too expensive because of the number to independent sensors (and corresponding wiring) that would be needed to accurately map the pressure areas. Additionally, they do not have the intelligent algorithm for tracing the history of the patients' movements.

This approach is analogous to using a high resolution digital camera to locate the positioned an object instead of employing a few (3 or more) IR optical sensors and a triangulation algorithm.

In an NIH funded research grant^{[26]}, M. FRIEDMAN proposes to develop wireless wearable monitors to measure and record the average positions over the course of a day and range of motion of the thigh of nursing home residents who are at risk for developing pressure ulcers. The system records the events when the patient is moved voluntarily or by the attendant. Their main research focus is to use their motion sensor and wireless transmitter to determine if variations in the timeliness of scheduled repositioning (turning in bed and restraint release from chairs) correlates with variations in pressure ulcer prevention effectiveness.

In 2003, an American Paraplegia Society paper^{[2]} suggested using the GPS (Global Positioning System) to determine if the individual has been motionless for a predetermined period of time. This approach is complex and has numerous disadvantages such as restrictions in the GPS signal reach within the buildings, spatial resolution of coordinates extracted from GPS signals, etc. To overcome some of these limitations, the authors proposed using DGPS (Differential GPS) with indoor transmitters, which adds to the complexity of the system. It is not self contained and relies on other sources.

We do not aim to replace the attendant, whom we regard as being vital to the care function for a mobility-impaired individual. We propose an inexpensive device and method for the prevention of bedsore injuries, which can regulate the intervention of the attendant, making it “interrupt-driven” and therefore freeing the attendant to perform other tasks without fear of overlooking the needed intervention. In addition to helping the conscientious attendant perform his/her duties more efficiently, this approach also identifies negligence, which is unfortunately casting an increasing aspersion on the health care industry today.

The principle of operation of the device is to characterize and monitor motion of the individual, and through the imposition of the movement thresholds, trigger events that correspond to the attainment of the necessary motion (required frequency and spatial extent) to prevent bedsores.

As another aspect and application, this invention enables the attendant to monitor stability in mentally-impaired and or intoxicated individuals by tracking the trajectory of their center of gravity during standing. This is helpful in the evaluating the degree of mental awareness and quantizing the course of treatment or therapy. The system may also be use to determine the degree of soberness in intoxicated individual. In this application, the apparatus may be used as alcohol consumption detection device that can more accurately determine the degree intoxication than the customary breathalyzer device currently used by law enforcement that simply measures the percentage of blood alcohol level. In this application, our invention may save thousands of deaths in traffic accidents alone.

The apparatus in this invention also has many applications in physical and occupational therapy and mental training (biofeedback), in sports and athletic training, sports paraphernalia design, etc.

### SUMMARY OF THE INVENTION

According to the principles of the present invention, an apparatus and the associated method provided that performs real-time monitoring and tracking of the dynamics of the center of pressure (which is equivalent to the projection of the center of gravity) on a platform upon which the individual is supported. This method is self-contained and does not require external signal sources or triangulation techniques for position monitoring. In one physical embodiment, the purpose is to determine when repositioning is needed, with the objective of alerting a healthcare attendant, locally or remotely through wired or wireless means. The alert status is maintained until the patient is repositioned adequately. Because the system decides on the time interval between repositionings as well as the extent of repositioning, the need for a skilled nurse is reduced or eliminated, allowing the health care task to be adequately performed by a person with no special skills or training, such as a family member of the patient. Last but not least, the system lends itself readily to telemedicine applications such as remote monitoring over the telephone network or the Internet, if desired.

The principal objective of this embodiment of our invention is to provide a lack-of-movement alarm for bedsore prevention for wheelchair bound, bed ridden and other immobile patients. However the device can be used for collateral applications such as rehabilitation and physical therapy of recovering stroke patients, patients recovering from hip and knee surgery, etc. The same hardware infrastructure lends itself quite naturally, with the appropriate software, to manifest a balance training function that could be part and parcel of a physical therapy program. Lastly, it may be coupled with actuators to automatically reposition the patient when necessary, significantly reducing the physical burden on the attendant and making his job that much easier. As another embodiment this application the system allows monitoring the required repositioning of bed-ridden patients such as stoke patients.

In another embodiment, this invention enables the attendant to monitor stability in mentally-impaired and/or intoxicated individuals by tracking the trajectory of their center of gravity while standing. This is helpful in the evaluating the degree of mental awareness and quantizing the course of treatment or therapy. The system may also be used to determine the degree of sobriety of a potentially intoxicated individual. In this application, the apparatus may be used to as alcohol consumption detection device that can more accurately determine the degree intoxication the customary breathalyzer device currently used by law enforcement that simply measures the percentage of blood alcohol level. In this application, our invention may save thousands of deaths in traffic accidents alone.

In yet another embodiment, the system described by the present invention may be incorporated in the driver's seat or the back of the driver's seat of the deriver in motor vehicles and be trained to alert the driver if he/she dozes off during driving. In similar embodiment, the system may be used to quantitatively analyze the results of crash test in automobiles by analyzing the magnitude and the center of the forces exerted by the would-be driver.

Other embodiments are possible for other useful applications. Some typical applications are briefly listed below:

The apparatus in this invention also has many applications in physical and occupational therapy and mental training (biofeedback), in sports and athletic training, sports paraphernalia design, etc.

The system has significant other applications and markets outside of healthcare and law enforcement, such as in sports (e.g., golf swing training, baseball batting practice), gymnastics, yoga, dance/ballet, and a host of other areas.

### BRIEF DESCRIPTION OF THE DRAWINGS

A more complete understanding of the present invention may be obtained from consideration of the following detailed description of the invention in conjunction with the drawing, with like elements referenced with like references, in which:

FIG. 1 shows a simplified block diagram of the System in an Embodiment for Displaying the Trajectory of Motion of Center of Pressure (COP) for a Wheelchair-bound Patient.

FIG. 2 shows a simplified Center of gravity (COG) Force Plate embodiment.

FIG. 3 is an illustration of how COP is calculated in a one-dimensional space (Cross Sectional View).

FIG. 4 is an illustration of how COP is calculated in a two-dimensional space (Top View).

FIG. 5 shows a typical Output Display Graphics for Motion Monitoring

FIG. 6 shows the application of COG Plate for wheelchair-bound individuals (Cross-sectional View).

FIG. 7 shows the application COG Plate for bedridden individuals.

(Cross sectional View).

FIG. 8 shows a typical embodiment of the present invention for monitoring the state of mental stability for mentally-impaired in patients or degree if intoxication in intoxicated individuals.

FIG. 9 depicts some of the applications for which the present invention may be adapted.

### DETAILED DESCRIPTION OF THE INVENTION

A more complete understanding of the present invention can be obtained in view detailed description of the illustrative figures.

System Overview

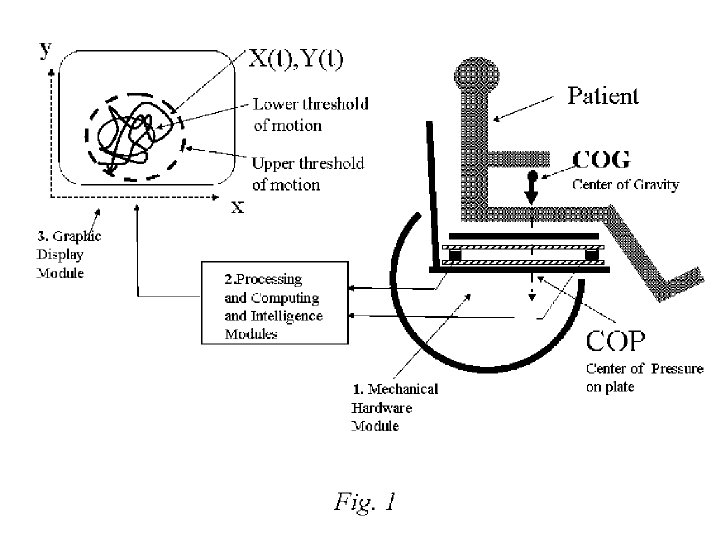

Accordingly, FIG. 1 represents an embodiment of the system adapted for monitoring a wheelchair bound patient for prevention of pressure ulcers, consisting of: a mechanical hardware module capable of producing electrical response on detection of movement of a subject (in this case a patient in a wheelchair); an electronic processing module capable of interpreting the electrical response of the hardware module into meaningful parameterization of the motion; and an output module that provides graphical (and optionally, audible) indication of the motion, and other inferential output.

Basic Principle

The basic principle of the system is to rely on the motion of the center of gravity (COG) of a patient as an indicator of body movement. This principle has been exploited in earlier work by one of the inventors in the study of human gaits at the Ohio State University's Human gait Laboratory^{[28]}.

The success of the method is predicated on being able to distinguish legitimate motion from “false positive” indications. An immediately apparent issue is that lateral translation (with no change in the pressure points) can potentially masquerade as a valid repositioning. To avoid this problem, the motion-detection algorithms process higher-order moments in addition to the basic COG calculation so that simple lateral motions can be distinguished from proper repositioning or rolling of the patient.

The hardware infrastructure of the system consists of a few (three or more) sensors that can be an integral part of the bed or chair (i.e., embedded in the legs), or part of a removable unit useable with existing beds or chairs. Without losing the essence of the argument, we will present our discussion in the context of the removable unit, which we will call the COG (for “Center of Gravity”) plate.

COG Plate

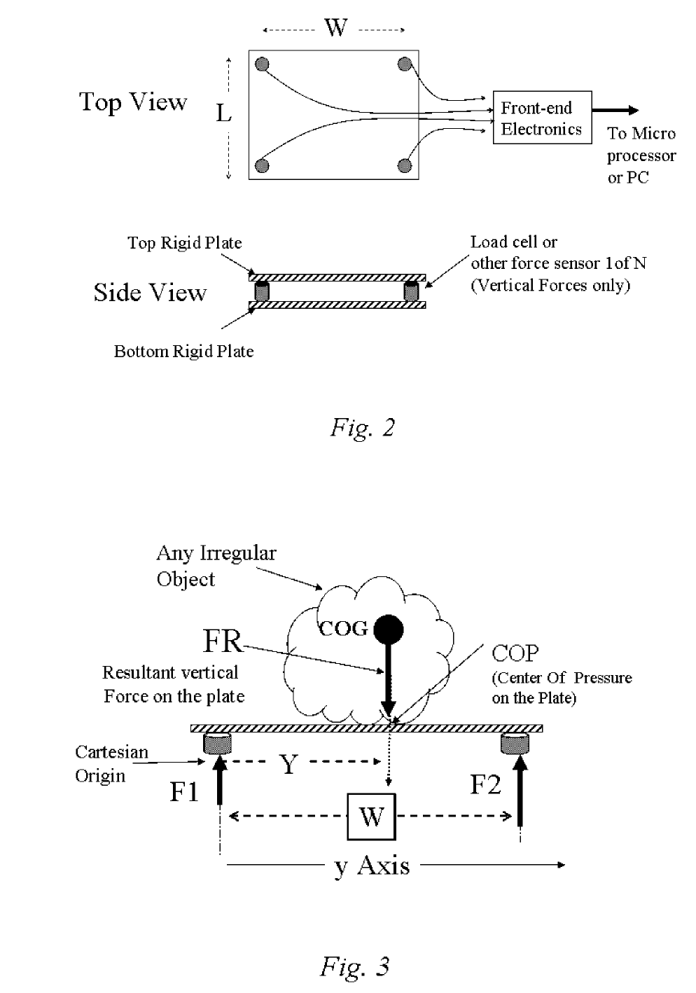

A COG Plate consisting of three or more force sensors sandwiched between two rigid plates is schematically depicted in FIG. 2 (which illustrates a plate with four sensors). The sensors may be any type of electromechanical or optical force sensors (such as piezo-resistive strain gauges or Fiber Bragg Grating stain sensors, etc.) that provide accurate electrical indication of the forces at the indicated points. This simple embodiment described here is important in ensuring a low-cost implementation.

Processor and Algorithm

The electrical output of each pressure sensor is fed to an electronic processing module, where it is amplified, conditioned, and digitized. The electronic module communicates the digitized information over a network (which could be wired or wireless) to a PC or processor, which uses the information to determine the COG (and higher-order moments relative to a fixed origin) of the load atop the plate assembly at fixed time intervals. The successive values of the calculated parameters are recorded in memory for analysis of the movement of the load, as well as for archiving the trajectories for later review, if necessary.

Finally, the processor runs an algorithm that operates on the motion data to realize the key function of our proposed system—the effective assessment of adequate position shift of the load, or lack thereof.

COG Calculation

In bed and wheelchair applications, the COG is the same as the Center Of Pressure (COP). In FIG. 3, we show how COP is calculated in one dimension. (Higher-order moments, such as the velocity and acceleration of the COP, are calculated similarly.)

Forces F1, F2 are measured by the two force sensors. Assuming that the plate is rigid, the location of COP in Y direction can be calculated from basic physics as follows:
Y=W*F2/(F1+F2) and total weight, FR=F1+F2.

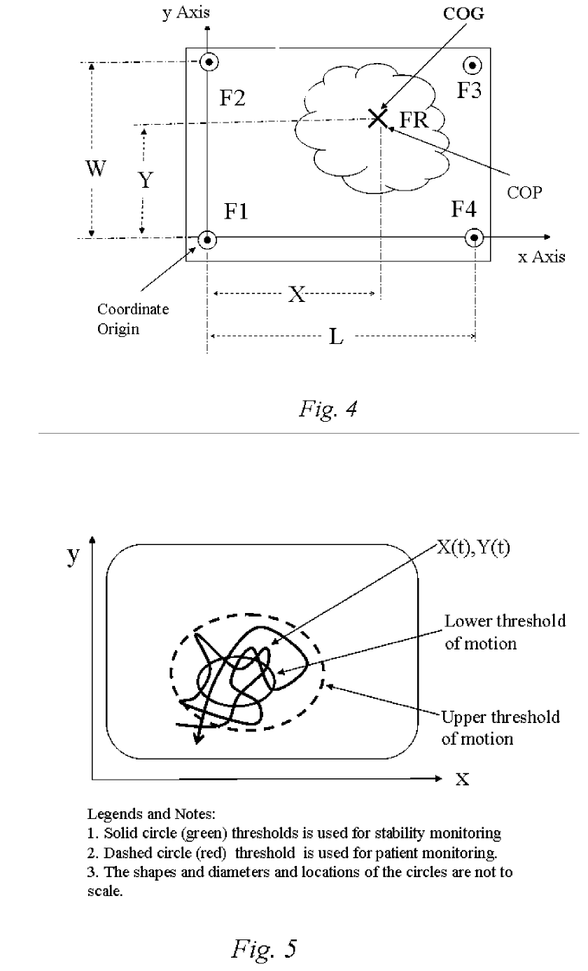

This computation may be easily extended to two dimensions as shown in FIG. 4. In this example, F1, F2, F3, F4 are measured by four force sensors; L and W are distances between force sensors; and X and Y represent the coordinates of the COP.

In a manner similar to the previous one-dimension calculations, the X and Y coordinates of the COP can be found as follows:
FR=F1+F2+F3+F4
X*FR=L*(F3+F4)
Y*FR=W*(F2+F3)
Thus,

$$X = L ( \frac{F 3 + F 4}{F 1 + F 2 + F 3 + F 4} )$$

$$Y = W ( \frac{F 2 + F 3}{F 1 + F 2 + F 3 + F 4} )$$

Output

If the forces are sampled at a fixed intervals of time T, then the trajectory (motion) of the center of pressure, COP, can be tracked displayed as a function of time X(t) and Y(t) as shown in FIG. 5. This figure shows a typical Output Display Graphics for Motion Monitoring. Assuming the speed of response of the force sensor is effectively instantaneous compared to the motion of the patient, this display provides the patient's COG dynamics information.

The graph shows a possible trajectory of COP as a function of time for a normal person: X(t), Y(t). The dashed (or red) circle (centered around instantaneous COP of a patient) shows a possible limit for normal motion. The solid (or green) circle (centered around instantaneous COP of a patient) shows a possible threshold for minimum motion. The minimum (solid or green shape) threshold would be used to detect absence of motion (to indicate a need for repositioning of a paralyzed patient, for example) while the maximum (dashed or red shape) threshold would be used to indicate excessive motion (such as a subject unable to maintain his balance during a sobriety test).

Higher-order moments, as well as the velocity and acceleration of the COP motion are also useful in analyzing the trajectory behavior. Slow, steady motion and rapid, jerky motion would be readily distinguishable using these higher-order moments.

Applications

The primary proposed application is in the field of healthcare, and will typically involve a person of limited mobility who is either bedridden or wheelchair-bound. The system has also other medical and none medical applications some of which are described herein.

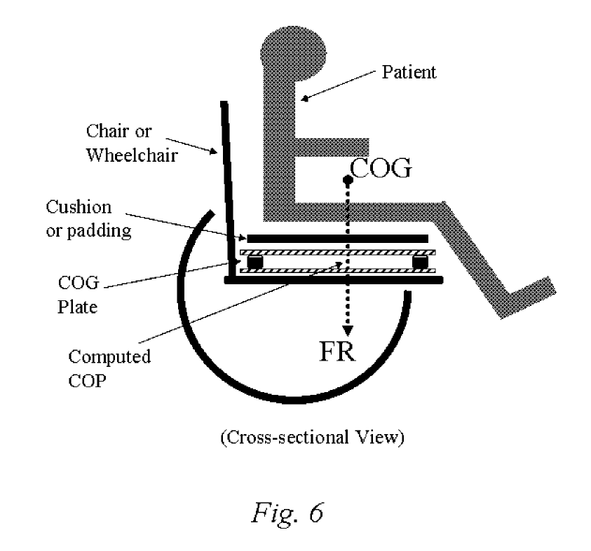

FIG. 6 symbolically illustrates the use of the COG plate in a wheelchair application. In the application a COG-plate is placed on the seat, but below the padding of a wheelchair-bound individual.

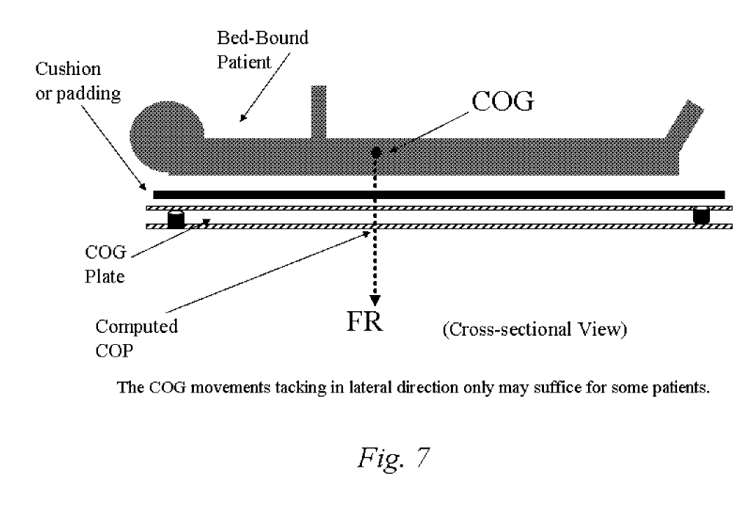

FIG. 7 shows the application of the COG Plate for bedridden individuals. Like the wheelchair case, here the bed platform is replaced by a cushioned COG plate. In this case, however, there may be a simplification in that the lateral movement alone might suffice.

As mentioned earlier, the sensors could (instead of being sandwiched between plates) be integrated into the legs of the bed or the frame of the wheelchair.

In the above, we presented a conceptual view of our method and devices to utilize COG monitoring and output to computer algorithms which determine whether it is necessary to issue an alert to reposition a mobility-impaired individual.

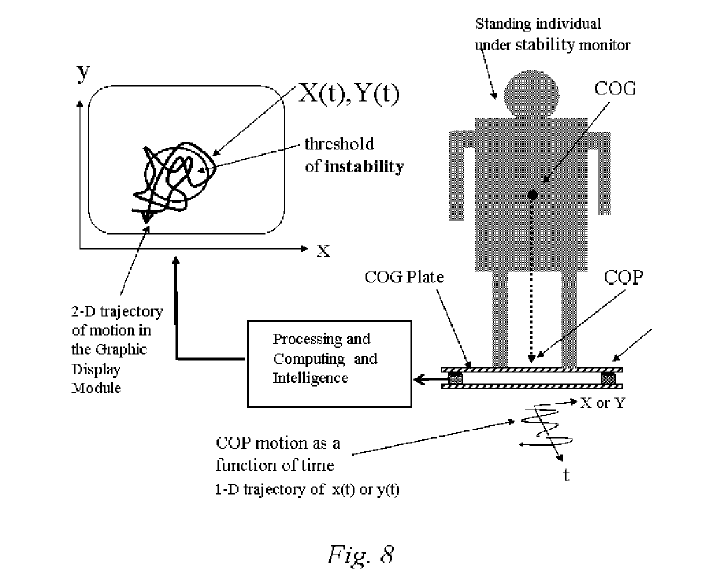

FIG. 8 shows a typical embodiment of the present invention for monitoring State of mental stability for mentally-impaired in patients or degree if intoxication in sobriety tests. In these applications the degree of individual's capability to stand still and in a stable manner is monitored. An alarm is set if the moments of COG during standing exceeds a preset threshold.

For DWI applications, law enforcement officers may use a COG plate apparatus to assess a driver's fitness to operate a motor vehicle in a manner which provides quantitative data which can be used in court.

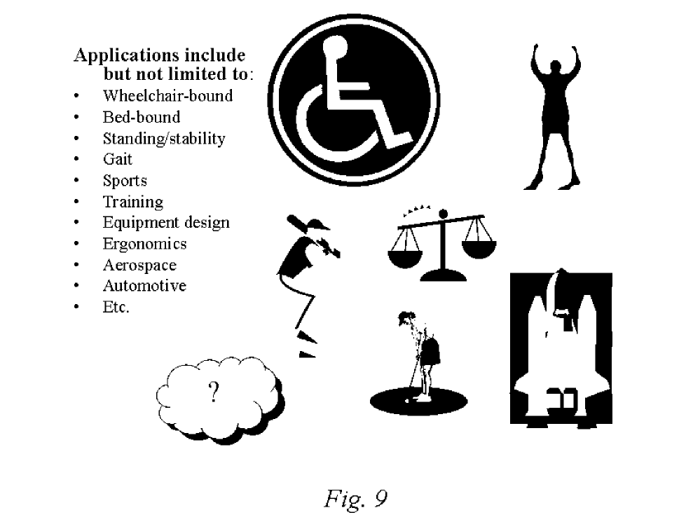

As stated earlier, other embodiments of this system have many applications in physical and occupational therapy, gait training, mental training (biofeedback), sports and athletic training, (e.g., golf swing training, baseball batting practice), gymnastics, yoga, dance/ballet, sports paraphernalia design, and a host of other areas, etc. FIG. 9 pictorially depicts some of the applications for which the present invention may be adapted.

It will be understood that the particular embodiments described above are only illustrative of the principles of the present invention, and that various modifications could be made by those skilled in the art without departing from the spirit and scope of the present invention. For example, the present invention may be advantageously used with other types of medical disability monitoring, therapy and training. For example for mentally-impaired children's gait training or stability training. Accordingly, the scope of the present invention is not limited only by the specifications listed above.

### REFERENCES AND PRIOR ART

References cited:
1. http://www.makoa.org/nscia/fact02.html
2. http://www.sci-info-pages.com/facts.html
3. http://www.americanheart.org/presenter.jhtml?identifier=4725
4. http://www.cdc.gov/nchs/fastats/stroke.htm
5. http://www.msif.org/en/ms_the_disease/quick_facts.html
6. http://www.unitedspinal.org/pages.php?catid=211&pageid=583&PHPSESSID=60e
7. Explanation Of Decubitus Ulcers Or Pressure Sores LDHP Medical Review Services Corp
8. Decubitus Ulcer Information and Wound Stages LDHP Medical Review Services Corporation Submitted December 1999
9. Spinal Cord Injury: Skin & Pressure Sores SCI-INFO Pages, May 2004
10. “Treating Pressure Sores” Consumer Version, Clinical Practice Guideline Number 15, December 1994, Department of Health and Human Services
11. Pressure Sores/Bed Sores & Maggots By Veronica Rose
12. BedSores . . . Easier to Prevent Than to Heal By: Clare Absher RN, BSN Publication of CarePathway, May 2004
13. US Patent 20040054303 Blanching response pressure sore detector apparatus and method
14. US Patent 20010020303 Mattress with bedsore preventing function
15. US patent 220010006646 METHOD AND SYSTEM FOR TREATMENT OF WOUNDS
16. U.S. Pat. No. 6,030,351 Pressure relief reminder and compliance system
17. U.S. Pat. No. 6,596,018 Mattress with bedsore preventing function
18. U.S. Pat. No. 5,584,084 Bed system having programmable air pump with electrically interlocking connectors
19. U.S. Pat. No. 4,530,122 Patient weight reliever apparatus
20. Dr. Ken Montgomery, CSIRO Textile and Fiber Technology, 03 9545 2330, 0418 235 103, Australia—Study confirms sheepskins prevent bed sores
21. Dyna-Care E4083 Pressure Pad System. Sammons Preston Products, Patent: Pressure adjustable mattress and seat to prevent decubitus ulcers. Inventor: Bayron A. Donzis, US Patent 20040064896 Contour and topography adjusting massaging mattress
22. http://www.progressivemedicalinc.com/air_mattresses.htm
23. http://www.linet.cz/document.asp?thema=2507&category=
24. WheelchairNet: Wheelchair Seats, Cushions and Positioning; http://www.wheelchairnet.org/WCN_ProdServ/Products/cushion.html
25. http://www.tekscan.com/medical/systems.html
26. Project Title: WIRELESS MONITOR TO PREVENT BEDSORES IN NURSING HOMES, PI Name: MARK B. FRIEDMAN, NIH Grant Number: 5R44AG012626-03
27. Prevention of Pressure Ulcers via Kinematic Global Positioning System Technology, Vidyajayawardena, M D, Meena Midha, M D, and Chand Samaratunga, PhD., American Paraplegia Society Conference, Paper #35, Las Vegas, Nev., September 2003
28. Balmaseda M. T., Fatehi M. T., Koozekanani, S. H., Reno C., Gerwin T., Hunter C., “Representation of Ground Reaction Forces as a function of the Position of the Center of Pressure During Gait,” Simulators, Vol. 16, No. 1, pp. 18-21, March 1985.

What is claimed is:

1. An apparatus for monitoring, tracking, recording, displaying, and performing calculations on the trajectory of the projection of the center of gravity of an object or individual, said apparatus consisting of:
a rigid force plate for supporting said object or individual and for measuring the vertical force components that said object or individual exerts on the support base and calculating the center of moment thereof by measuring the force components in at least three critical points under said object's support base;
an electronic amplifying and filtering means that receives signals relating to said force components from said various force sensors and providing them to a processing unit;
a sampling and digitizing means to sample said measured data values (COG data) at uniform or at non-uniform sampling intervals;
a processing unit and corresponding algorithms that computes the instantaneous coordinates of the center of pressure (the projection of the center of gravity) of said object, X(t) and Y(t), based on the values of the force sensors' output and their relative coordinate positions;
a means for processing, storing and recording said coordinate values, X and Y, as a function of time;
and a means for plotting and displaying the motion of said trajectory curves of the projection of the center of gravity of said object or individual on an electronic display unit such as a CRT or a computer monitor.

2. The apparatus of claim 1, wherein said force place consists of three or more force sensing elements, such as strain gauge sensors, fiber optic pressure sensors or other force sensing technologies, sandwiched between two rigid plates, wherein the electrical outputs of said sensors correlate to the force components at the location of the sensors.

3. The apparatus of claim 2, wherein the size and shape of said force plate is such that it encompasses the anticipated range of motion of said trajectory in order to indicate the stability of said object or individual (for determining balance, sobriety, etc.).

4. The apparatus of claims 1-3, wherein said display is programmed to plot a two-dimensional graph Y(X), of the coordinates X and Y of said trajectory of motion of said center of pressure, with time being implicit in the graph.

5. The apparatus of claim 4, wherein the time stamp is explicitly marked at critical points on said trajectory curve display.

6. The apparatus of claims 1-5, wherein said display is programmed to plot each of the two coordinates X and Y of said trajectory of motion of said center of pressure explicitly as a function of time X(t) and Y(t), in a manner similar to EKG graphs.

7. The apparatus of claims 1-6 wherein said processing unit includes means and/or algorithms for analyzing said COG data and triggering an alarm if said COG data exceeds predefined or adaptively calculated thresholds.

8. The apparatus of claims 1-6 wherein said processing unit includes means and/or algorithms for analyzing said COG data and triggering an alarm if said COG data fails to exceed predefined or adaptively calculated thresholds.

9. The apparatus of claims 7-8 wherein, said alarm is transmitted to at least one distant location via any communication means including, but not limited to, wireless networks, local-area networks (such as Ethernet), fiber optic networks, etc.

10. The apparatus of claims 1-9, with embodiment adapted to monitor the trajectory of the projection of the center of gravity of living objects or individuals standing on said force plate.

11. The apparatus of claim 10 adapted to monitor and evaluate the stability of mentally or physically impaired individuals.

12. The apparatus of claim 10 adapted and calibrated for sobriety or substance-abuse testing for use in DUI cases and other applications.

13. The apparatus of claims 1-9, with embodiment adapted to monitor the trajectory of the projection of the center of gravity of mobility-impaired individuals sitting in a wheelchair, wherein said force plate is adapted to fit in or on the frame of seat of said wheelchair.

14. The apparatus of claims 1-9, with embodiment adapted to monitor the trajectory of the projection of the center of gravity of mobility-impaired individuals lying in a bed, wherein said force plate is adapted to fit in or on said bed.

15. Apparatus of claims 1-9, with embodiment adapted to monitor the kinematics of a moving or rolling object, wherein said force plate is adapted to be sufficiently large to accommodate the anticipated range of motion of said moving or rolling object.

16. A method for monitoring, tracking, recording, displaying, and performing calculations on the trajectory of the projection of the center of gravity of an object or individual, using a typical embodiment consisting of:
a rigid force plate for supporting the said object or individual and for measuring the vertical force components that said object or individual exerts on the support base and calculating the center of moment thereof by measuring the force components in at least three critical points under said object's support base;
an electronic amplifying and filtering means that receives signals relating to said force components from said various force sensors and providing them to a processing unit;
a method and corresponding algorithms that computes the instantaneous coordinates of center of pressure or the projection of the center of gravity of said object (COG data), X(t) and Y(t), based on the values of said force sensors' output and their relative coordinate positions;
a method and a corresponding sampling and digitizing means to sample said COG data values at uniform or at non-uniform sampling intervals;
a method and a corresponding computing means for processing, storing and recording said coordinate values, X and Y, as a function of time;
and a method and corresponding means for plotting and displaying the motion of said trajectory curves of the projection of the center of gravity of said object or individual on an electronic display unit such as a CRT or a computer monitor.

17. The method of claim 16, wherein the size and shape of said force plate is such that it encompasses the anticipated range of motion of said trajectory in order to indicate the stability of said object or individual (for determining balance, sobriety, etc.).

18. The method of claims 16-17, wherein said display is programmed to plot a two-dimensional graph Y(X), of the coordinates X and Y of the trajectory of motion of the center of pressure of said object or individual.

19. The method of claims 16-17, wherein said display is programmed to plot each of the two coordinates X and Y of the trajectory of motion of the center of pressure of said object or individual explicitly as a function of time X(t) and Y(t), in a manner similar to EKG graphs.

20. The method of claims 16-19, wherein said processing unit includes means and/or algorithms for analyzing said COG data and triggering an alarm if said COG data exceeds predefined or adaptively calculated thresholds.

21. The method of claims 16-19 wherein said processing unit includes means and/or algorithms for analyzing said COG data and triggering an alarm if said COG data fails to exceed predefined or adaptively calculated thresholds.

22. The method of claims 20-21 wherein, said alarm is transmitted to at least one distant location via any communication means including, but not limited to, wireless networks, local-area networks (such as Ethernet), fiber optic networks, etc.

23. The method of claims 16-22, with embodiment adapted to monitor the trajectory of the projection of the center of gravity of living objects or individuals standing on said force plate.

24. The method of claim 23 adapted to monitor and evaluate the stability of mentally or physically impaired individuals.

25. The method of claim 24 adapted and calibrated for sobriety or substance-abuse testing for use in DUI cases and other applications.

26. Method of claims 16-22, with embodiment adapted to monitor the trajectory of the projection of the center of gravity of mobility-impaired individuals sitting in a wheelchair, wherein said force plate is adapted to fit in or on the frame of seat of said wheelchair.

27. Method of claims 16-22, with embodiment adapted to monitor the trajectory of the projection of the center of gravity of mobility-impaired individuals lying in a bed, wherein said force plate is adapted to fit in or on said bed.

28. Method of claims 16-22, with embodiment adapted to monitor the kinematics of a moving or rolling object, wherein said force plate is adapted to be sufficiently large to accommodate the anticipated range of motion of said moving or rolling object.
